(19) United States
(12) Patent Application Publication
(10) Pub. No.: US 2002/0188327 A1
(43) Pub. Date: Dec. 12, 2002

(54) Drug delivery for treatment of cardiac arrhythmia

(76) Inventor: Chester Struble, Eijsden (NL)

Correspondence Address: MEDTRONIC, INC., 710 MEDTRONIC PARKWAY NE, MS-LC340, MINNEAPOLIS, MN, 55432-5604, US

(21) Appl. No.: 09/842,148

(22) Filed: Apr. 26, 2001

(51) Int. Cl. A61N 1/362

(52) U.S. Cl. 607/9

(57) ABSTRACT

Techniques for the treatment of cardiac arrhythmia involve automatic administration of anti-arrhythmia drugs, infused into the patient's body by an implantable drug delivery system. The invention combines sophisticated techniques for discrimination among arrhythmia that originates in the atrium and arrhythmia that originates in the ventricle, and administering a drug treatment appropriate for the discriminated type of arrhythmia. The administration of anti-arrhythmia drugs may work in harmony with an implantable pacemaker.

### FIELD OF THE INVENTION

The present invention relates generally to detection and treatment of cardiac arrhythmia. More particularly, the present invention pertains to treatment of cardiac arrhythmia with anti-arrhythmia drugs.

### BACKGROUND

Any variation from the normal rhythm and sequence of excitation of the heart is called an arrhythmia. Arrhythmia may result from many factors, including interference with conduction of electrical signals, ectopic foci, or alterations in the activity of the sinoatrial (SA) node. Examples of arrhythmias include premature ventricular contraction (PVC), atrial flutter, atrial fibrillation or ventricular fibrillation. Arrhythmia may also include alterations in heart rate, such as tachycardia (rapid heart rate) or bradycardia (slow heart rate).

Some forms of arrhythmia are more serious than others. In addition, different forms of arrhythmia are treated in different ways. Detecting the form of arrhythmia is important in determining the seriousness of the arrhythmia and how the arrhythmia may best be treated.

Tachycardia, for example, may originate in the ventricles of the heart (ventricular tachycardia) or in the atria of the heart (supraventricular tachycardia, or SVT). SVT is a general term describing a number of conditions, including atrial fibrillation, atrioventricular (AV) nodal re-entrant tachycardia, and Wolff-Parkinson-White syndrome. The treatment for SVT is usually different from the treatment for ventricular tachycardia.

Some forms of arrhythmia may be treated by providing an electrical stimulation to the heart. Other forms of arrhythmia may be treated by introducing anti-arrhythmia drugs into the patient's body. Arrhythmia may also be treated with a combination of drugs and stimulation.

All forms of arrhythmia are not treated in the same way. Some drugs, such as ibutilide, may be indicated for atrial arrhythmia, while others, such as lidocaine, are indicated for ventricular arrhythmia.

When drug-based treatment of arrhythmia is indicated, the drug in many cases should be introduced shortly after the onset of the arrhythmia. The drug should also be delivered to an appropriate site, such as the patient's subclavian vein, superior vena cava (SVC) or fatty tissue.

An implantable drug delivery device represents an efficient means for rapid delivery of anti-arrhythmia drugs. Current anti-arrhythmia treatments, however, do not employ any implantable drug delivery devices. Furthermore, current anti-arrhythmia treatments do not include systems that automatically dispense drugs in response to arrhythmia, nor do the systems dispense drugs in response to a particular form of arrhythmia, such as atrial arrhythmia or ventricular arrhythmia.

Techniques for discriminating among the various types of arrhythmia are known in the art. Further, implantable drug delivery devices are known in the art. Examples of these techniques or devices may be found in the issued U.S. patents listed in Table 1 below.

TABLE 1

| U.S. Pat. No. | Inventor | Issue Date |
|---|---|---|
| 6,178,350 | Olson et al. | 01/23/2001 |
| 6,141,581 | Olson et al. | 10/31/2000 |
| 6,052,620 | Gillberg et al. | 04/18/2000 |
| 5,991,656 | Olson et al. | 11/23/1999 |
| 5,919,210 | Lurie et al. | 07/06/1999 |
| 5,855,593 | Olson et al. | 01/05/1999 |
| 5,800,498 | Obino et al. | 09/01/1998 |
| 5,755,761 | Obino | 05/26/1998 |
| 5,755,736 | Gillberg et al. | 05/26/1998 |
| 5,690,682 | Buscemi et al. | 11/25/1997 |
| 5,545,186 | Olson et al. | 08/13/1996 |
| 5,304,139 | Adams et al. | 04/19/1994 |
| 4,003,379 | Ellinwood, Jr. | 01/18/1977 |

All patents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the techniques of the present invention.

### SUMMARY OF THE INVENTION

The present invention has certain objects. That is, various embodiments of the present invention provide solutions to one or more problems existing in the prior art with respect to prior art pacemakers and arrhythmia treatments. In particular, it is an object of the present invention to provide an efficient means for rapid delivery of anti-arrhythmia drugs via one or more implantable drug delivery devices. A further object of the present invention is to provide techniques for automatically dispensing drugs in response to particular forms of arrhythmia, such as atrial arrhythmia or ventricular arrhythmia.

Another object of the invention is to provide treatment that meets each particular patient's needs. Accordingly, an object of the invention is to provide treatment appropriate for the patient and to allow treatment to be adjusted as needed. The patient's physician should be able to specify drug treatments for arrhythmia that best suit the patient. The patient's physician should further be able to observe of the patient's response to past treatment The present invention includes features to treat cardiac arrhythmia through automatic administration of anti-arrhythmia drugs as an alternative to, or in concert with, automatic electrical stimulation. The treatment appropriate for a patient's arrhythmia may be a function of the form of arrhythmia the patient is experiencing. Accordingly, the present invention combines sophisticated techniques for discrimination among forms of arrhythmia with administration of medicine from an implantable drug delivery device. This combination allows for the automatic administration of anti-arrhythmia drugs that are targeted for the specific form of arrhythmia experienced by the patient.

A further feature of the invention is that it is implantable in the patient's body. In particular, the drug delivery system may be implanted and may deliver drugs efficiently to the delivery sites. It is desirable that an expedient way be provided whereby drugs to the drug delivery system may be resupplied following implantation, because it is inefficient to perform surgery on the patient when the medications in the drug delivery system are exhausted. For this reason, one feature of the invention provides a convenient way to resupply drugs to the drug delivery system. In a typical embodiment, the drug delivery system includes one or more self-sealing membranes, permitting the resupply of drugs via a syringe.

The invention further includes features that support adaptation of drug delivery to the needs of each patient. The drug delivery system may deliver a variety of drugs according to the patient's needs, as determined by the patient's physician. The patient's physician may specify, for example, suitable anti-arrhythmia agents and suitable dosages. The invention further employs a programmable system in which drug delivery is a function of the detected form of arrhythmia and a function of instructions programmed by the physician.

In various embodiments, one or more of the features described above may provide a number of advantages. For example, the automatic administration of anti-arrhythmia drugs may work in harmony with an implantable pacemaker. The pacemaker may implement techniques for detection and discrimination of forms of arrhythmia. Accordingly, an advantage of the invention is that the invention establishes a cooperative system between an implantable pacemaker and an implantable drug delivery system. In one embodiment, drug administration is a function of signals from the pacemaker.

Another advantage provided by the invention is that the systems may be adapted to provide a treatment suitable for a patient's particular needs. The drugs and or dosages may be adjusted as needed. Furthermore, the effects of the treatment may be monitored, and additional treatment may be provided as appropriate.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a schematic view of an implantable medical device.

FIG. 2 shows the implantable medical device located in and near a heart.

FIG. 3 is a block diagram illustrating the constituent components of an implantable medical device.

FIG. 4 shows a pacemaker-cardioverter-defibrillator located in and near a heart.

FIG. 5 is a functional schematic diagram of one embodiment of an implantable medical device.

FIG. 6 is a diagram of a system including a pacing system and a drug delivery system.

FIG. 7 is a flow diagram illustrating techniques of the invention.

### DETAILED DESCRIPTION OF THE PREFERRED EMBODIMENTS

In the following detailed description of the preferred embodiments, reference is made to the accompanying drawings which form a part hereof, and in which is shown by way of illustration specific embodiments in which the invention may be practiced. It is to be understood that other embodiments may be utilized and structural or logical changes may be made without departing from the scope of the present invention. The following detailed description, therefore, is not to be taken in a limiting sense, and the scope of the present invention is defined by the appended claims.

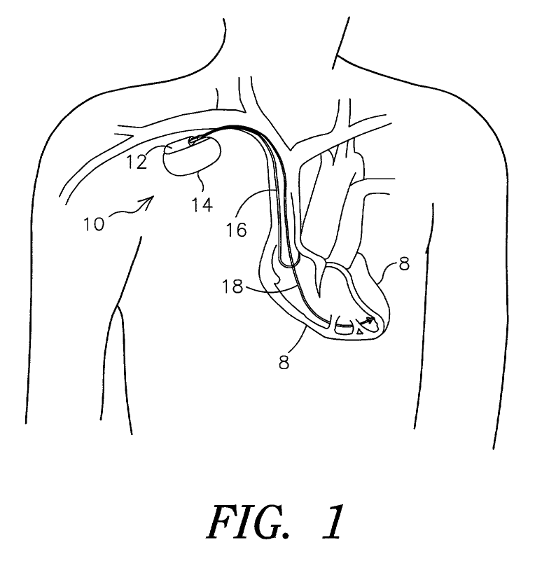

FIG. 1 is a simplified schematic view of one embodiment of implantable medical device (“IMD”) 10 of the present invention. IMD 10 shown in FIG. 1 is a pacemaker comprising at least one of pacing and sensing leads 16 and 18 attached to connector module 12 of hermetically sealed enclosure 14 and implanted near human or mammalian heart 8. Pacing and sensing leads 16 and 18 sense electrical signals attendant to the depolarization and repolarization of the heart 8, and further provide pacing pulses for causing depolarization of cardiac tissue in the vicinity of the distal ends thereof. Leads 16 and 18 may have unipolar or bipolar electrodes disposed thereon, as is well known in the art. Examples of IMD 10 include implantable cardiac pacemakers disclosed in U.S. Pat. No. 5,158,078 to Bennett et al., U.S. Pat. No. 5,312,453 to Shelton et al., or U.S. Pat. No. 5,144,949 to Olson, all hereby incorporated by reference herein, each in its respective entirety.

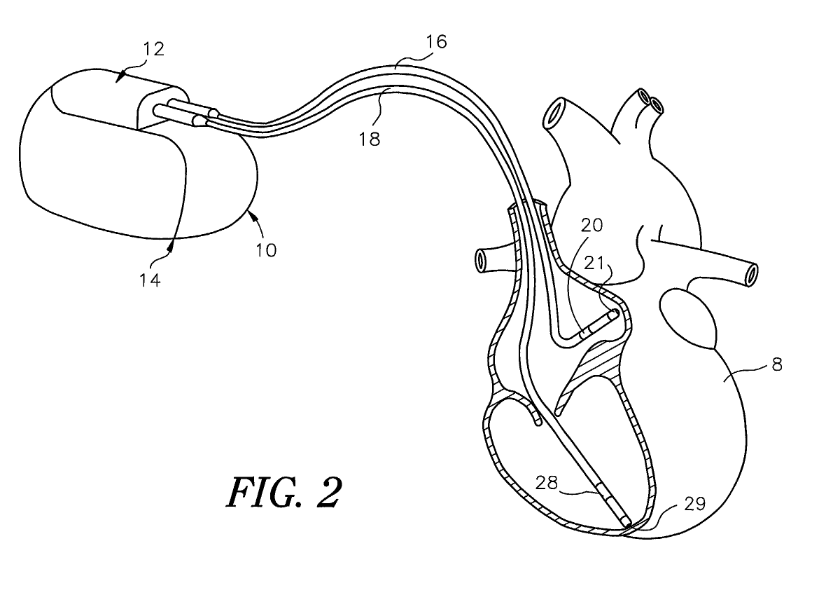

FIG. 2 shows connector module 12 and hermetically sealed enclosure 14 of IMD 10 located in and near human or mammalian heart 8. Atrial and ventricular pacing leads 16 and 18 extend from connector module 12 to the right atrium and ventricle, respectively, of heart 8. Atrial electrodes 20 and 21 disposed at the distal end of atrial pacing lead 16 are located in the right atrium. Ventricular electrodes 28 and 29 disposed at the distal end of ventricular pacing lead 18 are located in the right ventricle.

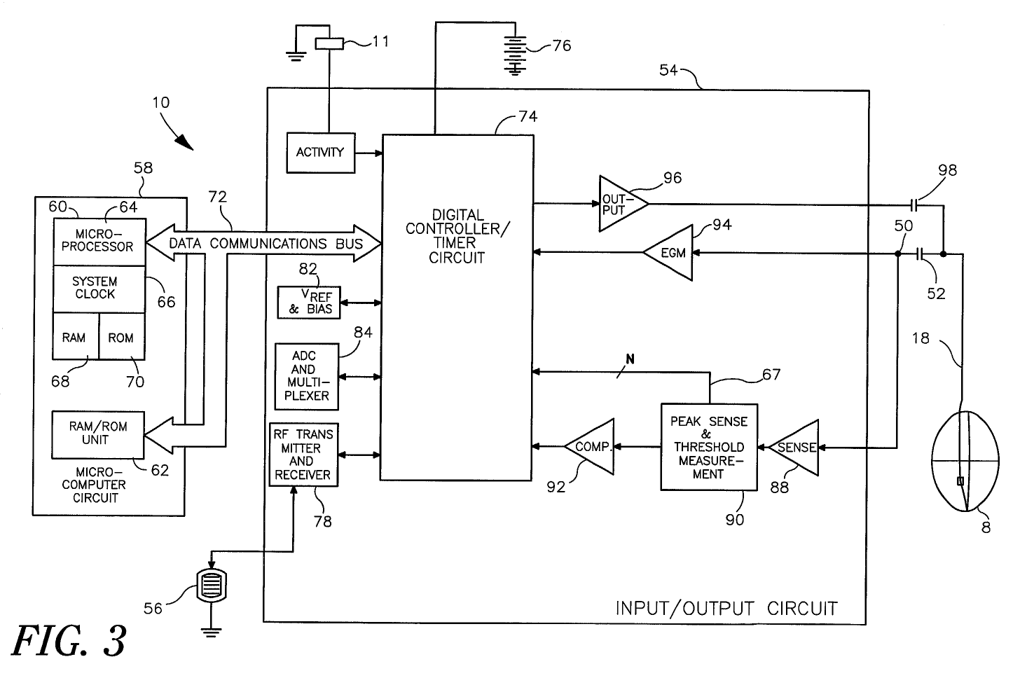

FIG. 3 shows a block diagram illustrating the constituent components of IMD 10 in accordance with one embodiment of the present invention, where IMD 10 is a pacemaker having a microprocessor-based architecture. IMD 10 is shown as including activity sensor or accelerometer 11, which is preferably a piezoceramic accelerometer bonded to a hybrid circuit located inside enclosure 14 (shown in FIGS. 1 and 2). Activity sensor 11 typically (although not necessarily) provides a sensor output that varies as a function of a measured parameter relating to a patient's metabolic requirements. For the sake of convenience, IMD 10 in FIG. 3 is shown with lead 18 only connected thereto. However, it is understood that similar circuitry and connections not explicitly shown in FIG. 3 apply to lead 16 (shown in FIGS. 1 and 2).

IMD 10 in FIG. 3 is most preferably programmable by means of an external programming unit (not shown in the figures). One such programmer is the commercially available Medtronic Model 9790 programmer, which is microprocessor-based and provides a series of encoded signals to IMD 10, typically through a programming head which transmits or telemeters radio-frequency (RF) encoded signals to IMD 10. Such a telemetry system is described in U.S. Pat. No. 5,312,453 to Wyborny et al., hereby incorporated by reference herein in its entirety. The programming methodology disclosed in Wyborny et al.'s '453 patent is identified herein for illustrative purposes only. Any of a number of suitable programming and telemetry methodologies known in the art may be employed so long as the desired information is transmitted to and from the pacemaker.

As shown in FIG. 3, lead 18 is coupled to node 50 in IMD 10 through input capacitor 52. Activity sensor or accelerometer 11 is most preferably attached to a hybrid circuit located inside hermetically sealed enclosure 14 of IMD 10. The output signal provided by activity sensor 11 is coupled to input/output circuit 54. Input/output circuit 54 contains analog circuits for interfacing with heart 8, activity sensor 11, antenna 56 and circuits for the application of stimulating pulses to heart 8. The rate of heart 8 is controlled by software-implemented algorithms stored within microcomputer circuit 58.

Microcomputer circuit 58 preferably comprises on-board circuit 60 and off-board circuit 62. Circuit 58 may correspond to a microcomputer circuit disclosed in U.S. Pat. No. 5,312,453 to Shelton et al., hereby incorporated by reference herein in its entirety. On-board circuit 60 preferably includes microprocessor 64, system clock circuit 66 and on-board RAM 68 and read-only memory (ROM) 70. Off-board circuit 62 preferably comprises a RAM/ROM unit. On-board circuit 60 and off-board circuit 62 are each coupled by data communication bus 72 to digital controller/timer circuit 74. Microcomputer circuit 58 may comprise a custom integrated circuit device augmented by standard RAM/ROM components.

Electrical components shown in FIG. 3 are powered by an appropriate implantable battery power source 76 in accordance with common practice in the art. For the sake of clarity, the coupling of battery power to the various components of IMD 10 is not shown in the Figures.

Antenna 56 is connected to input/output circuit 54 to permit uplink/downlink telemetry through RF transmitter and receiver telemetry unit 78. By way of example, telemetry unit 78 may correspond to that disclosed in U.S. Pat. No. 4,566,063 issued to Thompson et al., hereby incorporated by reference herein in its entirety, or to that disclosed in the above-referenced '453 patent to Wyborny et al. It is generally preferred that the particular programming and telemetry scheme selected permit the entry and storage of cardiac rate-response parameters. The specific embodiments of antenna 56, input/output circuit 54 and telemetry unit 78 presented herein are shown for illustrative purposes only, and are not intended to limit the scope of the present invention.

Continuing to refer to FIG. 3, V_{REF} and Bias circuit 82 most preferably generates stable voltage reference and bias currents for analog circuits included in input/output circuit 54. Analog-to-digital converter (ADC) and multiplexer unit 84 digitizes analog signals and voltages to provide “real-time” telemetry intracardiac signals and battery end-of-life (EOL) replacement functions. Operating commands for controlling the timing of IMD 10 are coupled from microprocessor 64 via data bus 72 to digital controller/timer circuit 74, where digital timers and counters establish the overall escape interval of the IMD 10 as well as various refractory, blanking and other timing windows for controlling the operation of peripheral components disposed within input/output circuit 54.

Digital controller/timer circuit 74 is preferably coupled to sensing circuitry, including sense amplifier 88, peak sense and threshold measurement unit 90 and comparator/threshold detector 92. Circuit 74 is further preferably coupled to electrogram (EGM) amplifier 94 for receiving amplified and processed signals sensed by lead 18. Sense amplifier 88 amplifies sensed electrical cardiac signals and provides an amplified signal to peak sense and threshold measurement circuitry 90, which in turn provides an indication of peak sensed voltages and measured sense amplifier threshold voltages on multiple conductor signal path 67 to digital controller/timer circuit 74. An amplified sense amplifier signal is also provided to comparator/threshold detector 92. By way of example, sense amplifier 88 may correspond to that disclosed in U.S. Pat. No. 4,379,459 to Stein, hereby incorporated by reference herein in its entirety.

The electrogram signal provided by EGM amplifier 94 is employed when IMD 10 is being interrogated by an external programmer to transmit a representation of a cardiac analog electrogram. See, for example, U.S. Pat. No. 4,556,063 to Thompson et al., hereby incorporated by reference herein in its entirety. Output pulse generator 96 provides amplified pacing stimuli to patient's heart 8 through coupling capacitor 98 in response to a pacing trigger signal provided by digital controller/timer circuit 74 each time either (a) the escape interval times out, (b) an externally transmitted pacing command is received, or (c) in response to other stored commands as is well known in the pacing art. By way of example, output amplifier 96 may correspond generally to an output amplifier disclosed in U.S. Pat. No. 4,476,868 to Thompson, hereby incorporated by reference herein in its entirety.

The specific embodiments of sense amplifier 88, output pulse generator 96 and EGM amplifier 94 identified herein are presented for illustrative purposes only, and are not intended to be limiting in respect of the scope of the present invention. The specific embodiments of such circuits may not be critical to practicing some embodiments of the present invention so long as they provide means for generating a stimulating pulse and are capable of providing signals indicative of natural or stimulated contractions of heart 8.

In some preferred embodiments of the present invention, IMD 10 may operate in various non-rate-responsive modes, including, but not limited to, DDD, DDI, VVI, VOO and VVT modes. In other preferred embodiments of the present invention, IMD 10 may operate in various rate-responsive modes, including, but not limited to, DDDR, DDIR, VVIR, VOOR and VVTR modes. Some embodiments of the present invention are capable of operating in both non-rate-responsive and rate responsive modes. Moreover, in various embodiments of the present invention IMD 10 may be programmably configured to operate so that it varies the rate at which it delivers stimulating pulses to heart 8 in response to one or more selected sensor outputs being generated. Numerous pacemaker features and functions not explicitly mentioned herein may be incorporated into IMD 10 while remaining within the scope of the present invention.

The present invention is not limited in scope to single-sensor or dual-sensor pacemakers, and is not limited to IMD's comprising activity or pressure sensors only. Nor is the present invention limited in scope to single-chamber pacemakers, single-chamber leads for pacemakers or single-sensor or dual-sensor leads for pacemakers. Thus, various embodiments of the present invention may be practiced in conjunction with one or more leads or with multiple-chamber pacemakers, for example. At least some embodiments of the present invention may be applied equally well in the contexts of single-, dual-, triple- or quadruple- chamber pacemakers or other types of IMD's. See, for example, U.S. Pat. No. 5,800,465 to Thompson et al., hereby incorporated by reference herein in its entirety, as are all U.S. Patents referenced therein.

IMD 10 may also be a pacemaker-cardioverter-defibrillator (“PCD”) corresponding to any of numerous commercially available implantable PCD's. Various embodiments of the present invention may be practiced in conjunction with PCD's such as those disclosed in U.S. Pat. No. 5,545,186 to Olson et al., U.S. Pat. No. 5,354,316 to Keimel, U.S. Pat. No. 5,314,430 to Bardy, U.S. Pat. No. 5,131,388 to Pless, and U.S. Pat. No. 4,821,723 to Baker et al., all hereby incorporated by reference herein, each in its respective entirety.

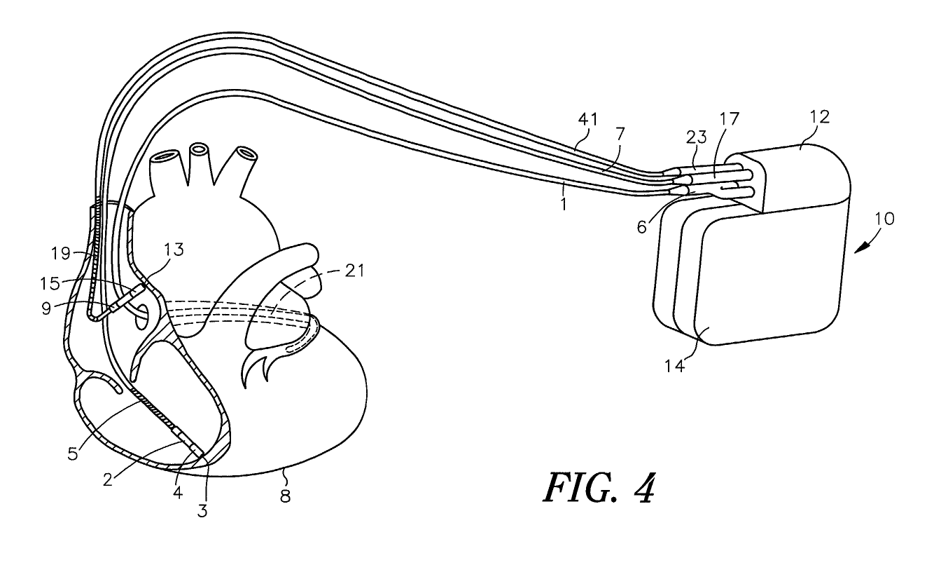

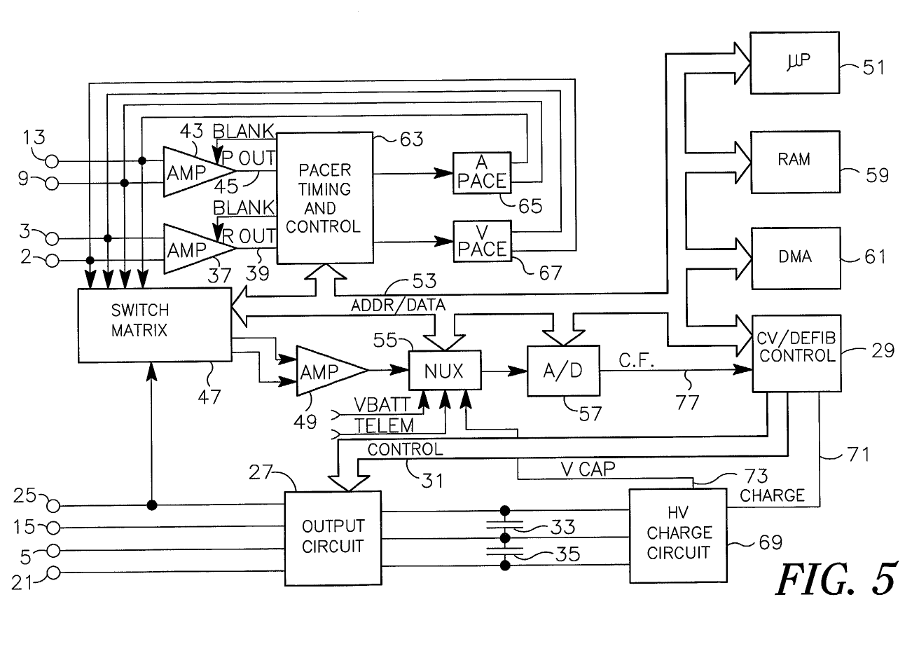

FIGS. 4 and 5 illustrate one embodiment of IMD 10 and a corresponding lead set of the present invention, where IMD 10 is a PCD. In FIG. 4, the ventricular lead takes the form of leads disclosed in U.S. Pat. Nos. 5,099,838 and 5,314,430 to Bardy, and includes an elongated insulative lead body 1 carrying three concentric coiled conductors separated from one another by tubular insulative sheaths. Located adjacent the distal end of lead 1 are ring electrode 2, extendable helix electrode 3 mounted retractably within insulative electrode head 4 and elongated coil electrode 5. Each of the electrodes is coupled to one of the coiled conductors within lead body 1. Electrodes 2 and 3 are employed for cardiac pacing and for sensing ventricular depolarizations. At the proximal end of the lead is bifurcated connector 6 which carries three electrical connectors, each coupled to one of the coiled conductors. Elongated coil electrode 5, which is a defibrillation electrode 5, may be fabricated from platinum, platinum alloy or other materials known to be usable in implantable defibrillation electrodes and may be about 5 cm in length.

The atrial/SVC lead shown in FIG. 4 includes elongated insulative lead body 7 carrying three concentric coiled conductors separated from one another by tubular insulative sheaths corresponding to the structure of the ventricular lead. Located adjacent the J-shaped distal end of the lead are ring electrode 9 and extendable helix electrode 13 mounted retractably within an insulative electrode head 15. Each of the electrodes is coupled to one of the coiled conductors within lead body 7. Electrodes 13 and 9 are employed for atrial pacing and for sensing atrial depolarizations. Elongated coil electrode 19 is provided proximal to electrode 9 and coupled to the third conductor within lead body 7. Electrode 19 preferably is 10 cm in length or greater and is configured to extend from the SVC toward the tricuspid valve. In one embodiment of the present invention, approximately 5 cm of the right atrium/SVC electrode is located in the right atrium with the remaining 5 cm located in the SVC. At the proximal end of the lead is bifurcated connector 17 carrying three electrical connectors, each coupled to one of the coiled conductors.

The coronary sinus lead shown in FIG. 4 assumes the form of a coronary sinus lead disclosed in the above cited '838 patent issued to Bardy, and includes elongated insulative lead body 41 carrying one coiled conductor coupled to an elongated coiled defibrillation electrode 24. Electrode 24, illustrated in broken outline in FIG. 4, is located within the coronary sinus and great vein of the heart. At the proximal end of the lead is connector plug 23 carrying an electrical connector coupled to the coiled conductor. Elongated coil defibrillation electrode 24 may be about 5 cm in length.

IMD 10 is shown in FIG. 4 in combination with leads 1, 7 and 41, and lead connector assemblies 23, 17 and 6 inserted into connector module 12. Optionally, insulation of the outward facing portion of housing 14 of IMD 10 may be provided using a plastic coating such as parylene or silicone rubber, as is employed in some unipolar cardiac pacemakers. The outward facing portion, however, may be left uninsulated or some other division between insulated and uninsulated portions may be employed. The uninsulated portion of housing 14 serves as a subcutaneous defibrillation electrode to defibrillate either the atria or ventricles. Lead configurations other that those shown in FIG. 4 may be practiced in conjunction with the present invention, such as those shown in U.S. Pat. No. 5,690,686 to Min et al., hereby incorporated by reference herein in its entirety.

FIG. 5 is a functional schematic diagram of one embodiment of IMD 10 of the present invention. This diagram should be taken as exemplary of the type of device in which various embodiments of the present invention may be embodied, and not as limiting, as it is believed that the invention may be practiced in a wide variety of device implementations, including cardioverter and defibrillators which do not provide anti-tachycardia pacing therapies.

IMD 10 is provided with an electrode system. If the electrode configuration of FIG. 4 is employed, the correspondence to the illustrated electrodes is as follows. Electrode 25 in FIG. 5 includes the uninsulated portion of the housing of IMD 10. Electrodes 25, 15, 24 and 5 are coupled to high voltage output circuit 27, which includes high voltage switches controlled by CV/defib control logic 29 via control bus 31. Switches disposed within circuit 27 determine which electrodes are employed and which electrodes are coupled to the positive and negative terminals of a capacitor bank (which includes capacitors 33 and 35) during delivery of defibrillation pulses.

Electrodes 2 and 3 are located on or in the ventricle of the patient and are coupled to the R-wave amplifier 37, which preferably takes the form of an automatic gain controlled amplifier providing an adjustable sensing threshold as a function of the measured R-wave amplitude. A signal is generated on R-out line 39 whenever the signal sensed between electrodes 2 and 3 exceeds the present sensing threshold.

Electrodes 9 and 13 are located on or in the atrium of the patient and are coupled to the P-wave amplifier 43, which preferably also takes the form of an automatic gain controlled amplifier providing an adjustable sensing threshold as a function of the measured P-wave amplitude. A signal is generated on P-out line 45 whenever the signal sensed between electrodes 9 and 13 exceeds the present sensing threshold. The general operation of R-wave and P-wave amplifiers 37 and 43 may correspond to that disclosed in U.S. Pat. No. 5,117,824 to Keimel et al., hereby incorporated by reference herein in its entirety.

Switch matrix 47 is used to select which of the available electrodes are coupled to wide band (0.5-200 Hz) amplifier 49 for use in digital signal analysis. Selection of electrodes is controlled by microprocessor 51 via data/address bus 53, which selections may be varied as desired. Signals from the electrodes selected for coupling to bandpass amplifier 49 are provided to multiplexer 55, and thereafter converted to multi-bit digital signals by A/D converter 57, for storage in random access memory (RAM) 59 under control of direct memory access circuit 61. Microprocessor 51 may employ digital signal analysis techniques to characterize the digitized signals stored in random access memory 59 to recognize and classify the patient's heart rhythm employing any of the numerous signal processing methodologies known to the art.

The remainder of the circuitry is dedicated to the provision of cardiac pacing, cardioversion and defibrillation therapies, and, for purposes of the present invention may correspond to circuitry known to those skilled in the art. The following exemplary apparatus is disclosed for accomplishing pacing, cardioversion and defibrillation functions. Pacer timing/control circuitry 63 preferably includes programmable digital counters which control the basic time intervals associated with DDD, VVI, DVI, VDD, AAI, DDI and other modes of single and dual chamber pacing well known to the art. Circuitry 63 also preferably controls escape intervals associated with anti-tachyarrhythmia pacing in both the atrium and the ventricle, employing any anti-tachyarrhythmia pacing therapies known to the art.

Intervals defined by pacing circuitry 63 include atrial and ventricular pacing escape intervals, the refractory periods during which sensed P-waves and R-waves are ineffective to restart timing of the escape intervals and the pulse widths of the pacing pulses. The durations of these intervals are determined by microprocessor 51, in response to stored data in memory 59 and are communicated to pacing circuitry 63 via address/data bus 53. Pacer circuitry 63 also determines the amplitude of the cardiac pacing pulses under control of microprocessor 51.

During pacing, escape interval counters within pacer timing/control circuitry 63 are reset upon sensing of R-waves and P-waves as indicated by a signals on lines 39 and 45, and in accordance with the selected mode of pacing on time-out trigger generation of pacing pulses by pacer output circuitry 65 and 67, which are coupled to electrodes 9, 13, 2 and 3. Escape interval counters are also reset on generation of pacing pulses and thereby control the basic timing of cardiac pacing functions, including anti-tachyarrhythmia pacing. The durations of the intervals defined by escape interval timers are determined by microprocessor 51 via data/address bus 53. The value of the count present in the escape interval counters when reset by sensed R-waves and P-waves may be used to measure the durations of R-R intervals, P-P intervals, P-R intervals and R-P intervals, which measurements are stored in memory 59 and used to detect the presence of tachyarrhythmias.

Microprocessor 51 most preferably operates as an interrupt driven device, and is responsive to interrupts from pacer timing/control circuitry 63 corresponding to the occurrence of sensed P-waves and R-waves and corresponding to the generation of cardiac pacing pulses. Those interrupts are provided via data/address bus 53. Any necessary mathematical calculations to be performed by microprocessor 51 and any updating of the values or intervals controlled by pacer timing/control circuitry 63 take place following such interrupts.

Detection of atrial or ventricular tachyarrhythmias, as employed in the present invention, may correspond to tachyarrhythmia detection algorithms known in the art. For example, the presence of an atrial or ventricular tachyarrhythmia may be confirmed by detecting a sustained series of short R-R or P-P intervals of an average rate indicative of tachyarrhythmia or an unbroken series of short R-R or P-P intervals. The rate of onset of the detected high rates, the stability of the high rates, and a number of other factors known in the art may also be measured at this time. Appropriate ventricular tachyarrhythmia detection methodologies measuring such factors are described in U.S. Pat. No. 4,726,380 issued to Vollmann, U.S. Pat. No. 4,880,005 issued to Pless et al., and U.S. Pat. No. 4,830,006 issued to Haluska et al., all incorporated by reference herein, each in its respective entirety. An additional set of tachycardia recognition methodologies is disclosed in the article “Onset and Stability for Ventricular Tachyarrhythmia Detection in an Implantable Pacer-Cardioverter-Defibrillator” by Olson et al., published in Computers in Cardiology, Oct. 7-10, 1986, IEEE Computer Society Press, pages 167-170, also incorporated by reference herein in its entirety. Atrial fibrillation detection methodologies are disclosed in Published PCT Application Ser. No. US92/02829, Publication No. WO92/8198, by Adams et al., and in the article “Automatic Tachycardia Recognition,” by Arzbaecher et al., published in PACE, May-June, 1984, pp. 541-547, both of which are incorporated by reference herein in their entireties.

In the event an atrial or ventricular tachyarrhythmia is detected and an anti-tachyarrhythmia pacing regimen is desired, appropriate timing intervals for controlling generation of anti-tachyarrhythmia pacing therapies are loaded from microprocessor 51 into the pacer timing and control circuitry 63, to control the operation of the escape interval counters therein and to define refractory periods during which detection of R-waves and P-waves is ineffective to restart the escape interval counters.

Alternatively, circuitry for controlling the timing and generation of anti-tachycardia pacing pulses as described in U.S. Pat. No. 4,577,633, issued to Berkovits et al., U.S. Pat. No. 4,880,005, issued to Pless et al., U.S. Pat. No. 4,726,380, issued to Vollmann et al., and U.S. Pat. No. 4,587,970, issued to Holley et al., all of which are incorporated herein by reference in their entireties, may also be employed.

In the event that generation of a cardioversion or defibrillation pulse is required, microprocessor 51 may employ an escape interval counter to control timing of such cardioversion and defibrillation pulses, as well as associated refractory periods. In response to the detection of atrial or ventricular fibrillation or tachyarrhythmia requiring a cardioversion pulse, microprocessor 51 activates cardioversion/defibrillation control circuitry 29, which initiates charging of high voltage capacitors 33 and 35 via charging circuit 69, under the control of high voltage charging control line 71. The voltage on the high voltage capacitors is monitored via VCAP line 73, which is passed through multiplexer 55 and in response to reaching a predetermined value set by microprocessor 51, results in generation of a logic signal on Cap Full (CF) line 77 to terminate charging. Thereafter, timing of the delivery of the defibrillation or cardioversion pulse is controlled by pacer timing/control circuitry 63. Following delivery of the fibrillation or tachycardia therapy microprocessor 51 returns the device to q cardiac pacing mode and awaits the next successive interrupt due to pacing or the occurrence of a sensed atrial or ventricular depolarization.

Several embodiments of appropriate systems for the delivery and synchronization of ventricular cardioversion and defibrillation pulses and for controlling the timing functions related to them are disclosed in U.S. Pat. No. 5,188,105 to Keimel, U.S. Pat. No. 5,269,298 to Adams et al., and U.S. Pat. No. 4,316,472 to Mirowski et al., hereby incorporated by reference herein, each in its respective entirety. Any known cardioversion or defibrillation pulse control circuitry is believed to be usable in conjunction with various embodiments of the present invention, however. For example, circuitry controlling the timing and generation of cardioversion and defibrillation pulses such as that disclosed in U.S. Pat. No. 4,384,585 to Zipes, U.S. Pat. No. 4,949,719 to Pless et al., or U.S. Pat. No. 4,375,817 to Engle et al., all hereby incorporated by reference herein in their entireties, may also be employed.

Continuing to refer to FIG. 5, delivery of cardioversion or defibrillation pulses is accomplished by output circuit 27 under the control of control circuitry 29 via control bus 31. Output circuit 27 determines whether a monophasic or biphasic pulse is delivered, the polarity of the electrodes and which electrodes are involved in delivery of the pulse. Output circuit 27 also includes high voltage switches which control whether electrodes are coupled together during delivery of the pulse. Alternatively, electrodes intended to be coupled together during the pulse may simply be permanently coupled to one another, either exterior to or interior of the device housing, and polarity may similarly be pre-set, as in current implantable defibrillators. An example of output circuitry for delivery of biphasic pulse regimens to multiple electrode systems may be found in the above-cited patent issued to Mehra and in U.S. Pat. No. 4,727,877 to Kallok, hereby incorporated by reference herein in its entirety.

An example of circuitry which may be used to control delivery of monophasic pulses is disclosed in U.S. Pat. No. 5,163,427 to Keimel, also incorporated by reference herein in its entirety. Output control circuitry similar to that disclosed in U.S. Pat. No. 4,953,551 to Mehra et al. or U.S. Pat. No. 4,800,883 to Winstrom, both incorporated by reference herein in their entireties, may also be used in conjunction with various embodiments of the present invention to deliver biphasic pulses.

Alternatively, IMD 10 may be an implantable nerve stimulator or muscle stimulator such as that disclosed in U.S. Pat. No. 5,199,428 to Obel et al., U.S. Pat. No. 5,207,218 to Carpentier et al., or U.S. Pat. No. 5,330,507 to Schwartz, or an implantable monitoring device such as that disclosed in U.S. Pat. No. 5,331,966 issued to Bennet et al., all of which are hereby incorporated by reference herein, each in its respective entirety. The present invention is believed to find wide application to any form of implantable electrical device for use in conjunction with electrical leads.

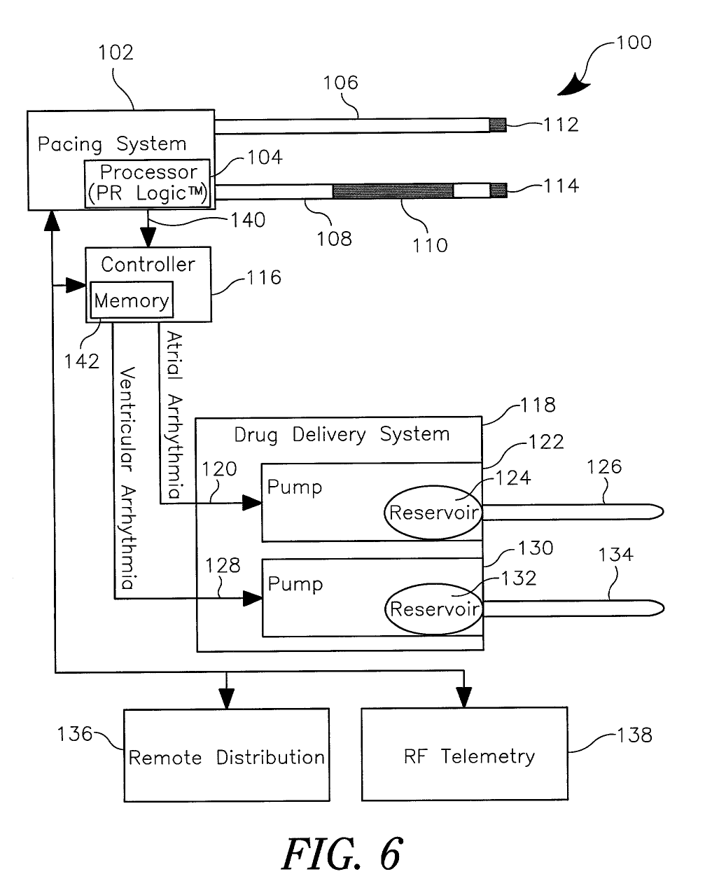

FIG. 6 shows a system 100 illustrating an embodiment of the invention. System 100 may be implantable in a human being or a mammal. System 100 includes pacing system 102, which paces heart 8. Pacing system 102 is coupled to leads 106 and 108, which extend to heart 8. Lead 106 may extend to the right atrium, and may include electrode 112. Lead 108 may extend to the right ventricle, and may include electrode 114 and defibrillation coil electrode 110.

Pacing system 102 may be one of the many forms of implantable medical devices described above. Atrial electrode 112 may correspond to any of electrodes 9, 13, 20 or 21 17 described above, ventricular electrode 114 may correspond to any of electrodes 2, 3, 28 and 29 described above, and defibrillation coil electrode 110 may correspond to elongated coil electrode 5 described above.

Heart 8 is monitored via leads 106 and 108. Electrical signals generated by heart 8 are sensed by electrodes 112 and 114, and are transmitted to pacing system 102. Electrodes 112 and 114 may be located, for example, in the right atrium and the right ventricle of heart 8, respectively. Pacing system 102 includes processor 104 configured to process signals received from electrodes 112 and 114, and to detect the presence of arrhythmia. In general, arrhythmia is detected by applying arrhythmia detection algorithms and applying predefined arrhythmia criteria to the patient's heart rhythm, as described above.

Processor 104 is further configured to discriminate between arrhythmia affecting the atrium, such as supraventricular tachycardia or SVT, and arrhythmia affecting the ventricle. Processor 104 may be realized as part of a PR Logic™ module, manufactured by and commercially available from Medtronic Inc. A PR Logic module receives atrial and ventricular electrical signals, and integrates rate detection data with information about conduction patterns, regularity and AV dissociation. The PR Logic module maintains a high sensitivity for ventricular arrhythmia, and also discriminates ventricular arrhythmia from atrial arrhythmia such as SVT. The PR Logic module may further generate arrhythmia signal 140, indicative of the discriminated arrhythmia. Pacing system 102 may be realized by, for example, AT-500™ pacemakers, or InSync-ICD™ or Gem DR™ implantable pacemaker-cardioverter-defibrillators, manufactured by and commercially available from Medtronic, Inc., each of which may include a PR Logic module.

After arrhythmia has been detected and processor 104 has discriminated the origin of the arrhythmia, processor 104 generates arrhythmia signal 140. Arrhythmia signal 140 may indicate that the patient is experiencing arrhythmia originating in an atrium and not arrhythmia originating in a ventricle, or vice versa. Arrhythmia signal 140 may include additional data pertaining to the arrhythmia. Additional data may include, for example, the heart rate. Additional data may also provide the results of further discrimination of the arrhythmia by processor 104, such as whether a detected atrial arrhythmia is a mild flutter or a more serious fibrillation.

Controller 116 receives arrhythmia signal 140 from pacing system 102. Controller 116 regulates the operation of drug delivery system 118 as a function of arrhythmia signal 140.

When arrhythmia signal 140 from processor 104 indicates the occurrence of atrial arrhythmia, controller 116 may evaluate whether arrhythmia signal 140 indicates treatment by anti-arrhythmia drug. When treatment by drug is indicated, controller 116 may send control signal 120 to pump 122. Pump 122 includes reservoir 124, which holds a drug useful for treatment of atrial arrhythmia. Reservoir 124 may hold, for example, digitalis or a beta blocker. Pump 122 dispenses the anti-arrhythmia drug from reservoir 124 to the patient's body via catheter 126.

Similarly, when the signal from processor 104 indicates the occurrence of ventricular arrhythmia, controller 116 may evaluate whether arrhythmia signal 140 indicates treatment by anti-arrhythmia drug. When treatment by drug is indicated, controller 116 may send control signal 128 to pump 130. Pump 130 includes reservoir 132, which holds a drug useful for treatment of ventricular arrhythmia. Reservoir 132 may hold, for example, amiodarone or lidocaine. Pump 130 dispenses the anti-arrhythmia drug from reservoir 132 to the patient's body via catheter 134.

The agents held by reservoirs 124 and 132 may be selected by the patient's physician, based upon the patient's particular needs. Anti-arrhythmia agents may include any of several classes of drugs, typically organized according to their properties. Class I includes sodium channel blockers, Class II includes beta blockers, Class III includes agents that prolong the refractory period by lengthening the action potential, and Class IV includes calcium channel blockers. These classes are non-exclusive, and a single drug, such as amiodarone, may belong to several classes. Although the present invention may be used to administer drugs having Class I, II, III and/or IV properties, the invention is not limited to use with such drugs. The present invention may be used to deliver drugs that do not fall in any of the classes, as well as new drugs that may be discovered.

Examples of implantable pumps include a number of SynchroMed™ pumps manufactured by and commercially available from Medtronic Inc. Pumps of this kind typically include self-sealing reservoirs that may be refilled by a needle and syringe, and need not be surgically removed when empty. The needle and syringe may also be used to drain a pump of one drug, flush the reservoir, and refilled the reservoir with a different drug. The pumps may further include a fill port (not shown in FIG. 6) that assists the medical personnel refilling the reservoir. The invention is not limited to use with SynchroMed pumps, however, and may be adapted for use with other models of implantable drug pumps.

Infusion apparatus, such as catheters 126 and 134, infuse drugs from reservoirs 124 and 132 to one or more infusion sites the patient's body. The infusion site may depend upon the drug being infused. Catheters 126 and 134 may dispense drugs at two or more infusion sites within the patient's body. For example, a catheter may deliver drugs to the patient's subclavian vein, or to the patient's SVC or to the patient's fatty tissue. If the patient has more than one catheter, the catheters need not deliver drugs to the same infusion site.

Controller 116, which regulates the operation of drug delivery system 118, may include memory 142. Memory 142 may be used to store therapy data, such as the amount of drug dispensed to the patient, the estimated amount of drug remaining in reservoirs 124 and 132, the number of dosages supplied, therapy trends, and so forth.

The patient's physician may access this data by input/output devices such as remote distribution link 136 or RF telemetry 138. Remote distribution link 136 provides a channel for downloading data from the patient over a telephone line or over the internet, for example. RF telemetry 138 provides immediate access to the data on a dedicated channel. Typically, a patient is required to visit the physician's office when data are to be downloaded via RF telemetry 138.

Input/output devices 136 and 138 allow a person such as the patient's physician to exchange information with pacing system 102 or controller 116. The information exchanged may include drug delivery data, pacing data, patient activity data, and other numbers, statistics or data.

Input/output devices 136 and 138 may also be used to program controller 116. Controller may access memory 142 to store the instructions or parameters programmed by the physician and to retrieve stored instructions or parameters. The physician may program, for example, minimum or maximum dosages, frequency of administration, and various other criteria for delivery of drugs. The physician may further program controller 116 with atrial arrhythmia criteria and ventricular arrhythmia criteria. Atrial and ventricular arrhythmia criteria may be employed by controller 116 to evaluate whether arrhythmia signal 140 indicates treatment by anti-arrhythmia drug, treatment by a combination of anti-arrhythmia drug and electric defibrillation, or no treatment at all.

The physician may further program controller 116 to act in concert with pacing system 102. A patient susceptible to acute episodes of ventricular fibrillation, for example, may benefit from having drug delivery system 118 and pacing system 102 acting in concert. When an acute episode of ventricular fibrillation is detected, controller 116 transmits control signal 128 to drug delivery system 118, causing drug pump 130 to deliver a bolus injection to the patient. After delivery of the bolus, a defibrillation pulse may be delivered to the patient via defibrillation coil electrode 110. In an exemplary operation, there may be a delay between infusion of the bolus and the defibrillation. The administration of a large dosage of medication prior to stimulation may aid in the success of the therapy.

The above example is offered for purposes of illustration, and the invention is not limited to the circumstances described. There are many situations in which treatment by drugs and by electrical stimulation may act in concert. The patient's physician may program system 100 to provide the therapy best designed to meet the patient's needs.

Controller 116 may be housed inside pacing system 102, or housed in drug delivery system 118, or may be a component separate from system 102 and 118. In addition, pacing system 102 and drug delivery system 118 may be housed together. In many cases, however, drug delivery system 118 may best deliver drugs from a site remote from pacing system 102.

Signals, data and programming may be transmitted among pacing system 102, controller 116, drug delivery system 118 and input/output devices 136 and 138 by hard wire, optical cable, wireless telemetry, or any combination thereof. In an implanted system, signals, data and programming may also be transmitted by modulated ultrasound, as described in U.S. Pat. No. 5,113,859 to Funke, hereby incorporated by reference herein in its entirety. The invention is not limited to any particular communication link among the components.

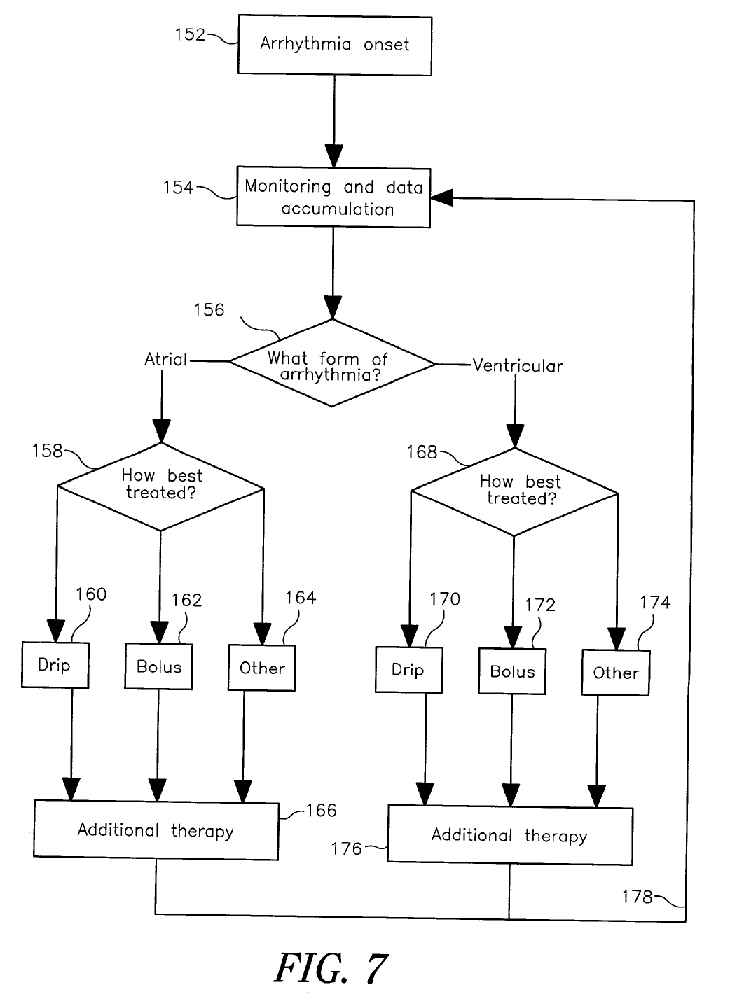

FIG. 7 is a flowchart illustrating techniques of the invention. Upon onset of arrhythmia (152), data pertinent to the patient's condition are collected. Collected data may include, for example, cardiac electrical activity detected by electrodes 112 and 114. The collected data are analyzed by PR Logic module 104, which discriminates among the forms of arrhythmia (156).

Based upon the collected data and instructions from the patient's physician, controller 116 selects the appropriate drug treatment (158, 168). For atrial arrhythmia, treatments may include infusion of medication by drip (160), bolus (162) or by other treatment (164). Similarly, ventricular arrhythmia may be treated by drip (170), bolus (172) or other treatment (174).

When indicated by the collected data and instructions from the patient's physician, controller 116 may provide, coordinate or cooperate in additional therapy (166, 176). Additional therapy may include, for example, electrical stimulation by pacing system 102 via defibrillation coil electrode 110.

Following the therapy, the patient's condition may be monitored to assess the results of the therapy. The results may be fed back (178), and may be used by pacing system 102 and/or controller 116 in the provision of further therapy.

The preceding specific embodiments are illustrative of the practice of the invention. It is to be understood, therefore, that other expedients known to those skilled in the art or disclosed herein may be employed without departing from the invention or the scope of the claims. For example, the present invention is presented with two drug pumps but the invention is not limited to two drug pumps. On the contrary, some patients may benefit from one pump but not from a second pump. The single pump may be activated when arrhythmia signal 140 indicates the patient is experiencing ventricular arrhythmia, for example, but will stand idle and not be activated when arrhythmia signal 140 indicates the patient is experiencing atrial arrhythmia.

Other patients may benefit from more than two drug pumps. Controller 116 may be programmed, for example, to dispense a first drug from a first drug pump in response to atrial arrhythmia, to dispense a second drug from a second drug pump in response to an onset of ventricular arrhythmia, and to dispense a third drug from a third drug pump as continuing treatment of the ventricular arrhythmia.

The invention further includes within its scope the methods of making and using the systems described above. These methods are not limited to the specific examples described above, but may be adapted to meet the needs of a particular patient. These and other embodiments are within the scope of the following claims.

In the claims, means-plus-functions clauses are intended to cover the recited structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

1. A system for treating cardiac arrhythmia, the system comprising:
a sensing lead configured to sense electrical signals attendant to the depolarization and repolarization of a heart;
a processor configured to receive the electrical signals, to detect cardiac arrhythmia from the electrical signals, to discriminate between an atrial arrhythmia and a ventricular arrhythmia as a function of the electrical signals, and to generate an arrhythmia signal as a function of the type of arrhythmia discriminated from the electrical signals; and
a drug delivery system configured to receive the arrhythmia signal, the drug delivery system comprising:
a first drug pump containing a first drug;
a second drug pump containing a second drug;
a first infusion apparatus coupled to the first drug pump; and
a second infusion apparatus coupled to the second drug pump,
wherein the drug delivery system is configured to activate the first drug pump to dispense the first drug via the first infusion apparatus when the arrhythmia signal is indicative of atrial arrhythmia, and
wherein the drug delivery system is configured to activate the second drug pump to dispense the second drug via the second infusion apparatus when the arrhythmia signal is indicative of ventricular arrhythmia.

2. The system of claim 1, further comprising a pacing system coupled to the processor, the pacing system including a pacing lead configured to provide pacing pulses to cardiac tissue.

3. The system of claim 1, wherein the system is implantable in a human body.

4. The system of claim 1, wherein the sensing lead is a first sensing lead, the system further comprising a second sensing lead configured to sense electrical signals attendant to the depolarization and repolarization of the heart, wherein one of the sensing leads is located in an atrium of the heart and the other of the sensing leads is located in a ventricle of the heart.

5. The system of claim 1, further comprising a controller, the controller configured to receive the arrhythmia signal, to generate a first control signal to activate the first drug pump when the arrhythmia signal is indicative of atrial arrhythmia, and to generate a second control signal to activate the second drug pump when the arrhythmia signal is indicative of ventricular arrhythmia.

6. The system of claim 5, further comprising memory, the controller interacting with the memory to access treatment instructions and parameters.

7. The system of claim 5, further comprising an input/output device coupled to the controller.

8. The system of claim 1, the drug delivery system further comprising:
a third drug pump containing a third drug; and
a third infusion apparatus coupled to the third drug pump;
wherein the drug delivery system is configured to activate the third drug pump as a function of the arrhythmia signal.

9. The system of claim 1, wherein the first drug is selected from the group consisting of digitalis and beta blockers.

10. The system of claim 1, wherein the second drug is selected from the group consisting of lidocaine and amiodarone.

11. A method of treating arrhythmia in a patient,
wherein a drug delivery system has been implanted in the patient, the drug delivery system comprising a first drug pump containing a first drug and a second drug pump containing a second drug, the method comprising:
receiving an arrhythmia signal indicative of one of atrial arrhythmia or ventricular arrhythmia;
activating the first drug pump to dispense the first drug when the arrhythmia signal is indicative of atrial arrhythmia, and
activating the second drug pump to dispense the second drug when the arrhythmia signal is indicative of ventricular arrhythmia.

12. The method of claim 11, further comprising:
monitoring the heart rhythm of the patient; and
generating an arrhythmia signal when the heart rhythm meets defined arrhythmia criteria.

13. The method of claim 11, further comprising:
selecting a first drug dosage;
activating the first drug pump to dispense the first drug dosage;
selecting a second drug dosage; and
activating the second drug pump to dispense the second drug dosage.

14. The method of claim 13, wherein a dosage comprises one of a drip dosage or a bolus dosage.

15. The method of claim 11, wherein the arrhythmia signal is further indicative of acute ventricular arrhythmia, the method further comprising:
activating the second drug pump to dispense a bolus of the second drug; and
applying a defibrillating pulse to the patient's heart.

16. The method of claim 15, wherein the defibrillating pulse is applied after the bolus is dispensed.

17. A system comprising:
a processor configured to receive the electrical signals from a heart and to generate an arrhythmia signal as a function of the type of electrical signals, the arrhythmia signal indicative of one of atrial arrhythmia or ventricular arrhythmia; and
a drug delivery system configured to receive the arrhythmia signal, the drug delivery system comprising a drug pump and an infusion apparatus coupled to the drug pump,
wherein the drug delivery system is configured to activate the drug pump to dispense the drug via the infusion apparatus when the arrhythmia signal is indicative of one of atrial arrhythmia and ventricular arrhythmia.

18. The system of claim 17, wherein the drug delivery system is configured to stand idle when the arrhythmia signal is indicative of the other of atrial arrhythmia and ventricular arrhythmia.

19. The system of claim 18, wherein the system is implantable in a human body.

20. The system of claim 17, further comprising a pacing system coupled to the processor.

21. The system of claim 17, further comprising a sensing lead configured to sense electrical signals attendant to the depolarization and repolarization of the heart.

22. The system of claim 17, further comprising a controller, the controller configured to receive the arrhythmia signal, to generate a control signal to activate the drug pump when the arrhythmia signal is indicative of one of atrial arrhythmia and ventricular arrhythmia.

23. A method for treatment of arrhythmia in a patient comprising:
implanting in the patient a sensing lead configured to sense electrical signals attendant to the depolarization and repolarization of the patient's heart;
implanting in the patient a processor configured to receive the electrical signals, to detect cardiac arrhythmia from the electrical signals and to discriminate between an atrial arrhythmia and a ventricular arrhythmia as a function of the electrical signals;
implanting in the patient a drug delivery system comprising a first drug pump containing a first drug and a second drug pump containing a second drug;
monitoring the patient's heart rhythm using the sensing lead and the processor;
upon detection of arrhythmia, determining the type of the arrhythmia using the processor;
responsive to atrial arrhythmia, activating the first drug pump to dispense the first drug to the patient; and
responsive to ventricular arrhythmia, activating the second drug pump to dispense the second drug to the patient.

24. The method of claim 23, further comprising implanting a pacing system, the pacing system including a pacing lead configured to provide pacing pulses for causing depolarization of cardiac tissue.

25. The method of claim 23, further comprising:
implanting in the patient a device configured to apply a defibrillating pulse to the patient's heart;
activating the first drug pump to dispense a bolus of the first drug; and
applying a defibrillating pulse to the patient's heart.

26. The method of claim 23, further comprising:
implanting in the patient a device configured to apply a defibrillating pulse to the patient's heart;
activating the second drug pump to dispense a bolus of the second drug; and
applying a defibrillating pulse to the patient's heart.

27. The method of claim 23, further comprising:
implanting in the patient a first infusion apparatus coupled to the first drug pump and a first infusion site; and
implanting in the patient a second infusion apparatus coupled to the second drug pump and a second infusion site.

28. The method of claim 27, wherein an infusion site is one of subclavian vein, superior vena cava and fatty tissue.

29. The method of claim 23, further comprising
implanting in the patient a controller configured to activate the first drug pump in response to atrial arrhythmia and to activate the second drug pump in response to ventricular arrhythmia.

30. The method of claim 29, further comprising programming the controller with atrial arrhythmia criteria.

31. The method of claim 29, further comprising programming the controller with ventricular arrhythmia criteria.

32. The method of claim 29, further comprising programming the controller with criteria for delivery of the first and second drugs, wherein criteria for delivery includes minimum dosage, maximum dosage and frequency of administration.

33. The method of claim 23, wherein the first drug pump includes a first reservoir containing the first drug, wherein the second drug pump includes a second reservoir containing the second drug, the method further comprising:
refilling the first reservoir with the first drug; and
refilling the second reservoir with the second drug.

34. The method of claim 33, wherein refilling a reservoir comprises refilling a reservoir using a needle and syringe.

35. A system for treating cardiac arrhythmia comprising:
a processor configured to detect cardiac arrhythmia, to discriminate between an atrial arrhythmia and a ventricular arrhythmia, and to generate an arrhythmia signal as a function of the type of arrhythmia;
a drug delivery system comprising a first drug pump containing a first drug, a second drug pump containing a second drug, a first infusion apparatus coupled to the first drug pump and a second infusion apparatus coupled to the second drug pump; and
a controller configured to receive the arrhythmia signal, to generate a first control signal to activate the first drug pump to dispense the first drug via the first infusion apparatus when the arrhythmia signal is indicative of atrial arrhythmia, and to generate a second control signal when the arrhythmia signal is indicative of atrial arrhythmia, and to generate a second control signal to activate the second drug pump to dispense the second drug via the second infusion apparatus when the arrhythmia signal is indicative of ventricular arrhythmia.

36. The system of claim 35, further comprising a sensing lead coupled to the processor, the sensing lead configured to sense electrical signals attendant to the depolarization and repolarization of a patient's heart.

37. The system of claim 35, further comprising a pacing system coupled to the processor, the pacing system including a pacing lead configured to provide pacing pulses for causing depolarization of cardiac tissue.

38. The system of claim 37, further comprising a device coupled to the pacing system configured to apply a defibrillating pulse to the cardiac tissue.

39. The system of claim 35, wherein the processor, drug delivery system and controller are implantable in a human body.

40. A system for treating cardiac arrhythmia comprising:
means for sensing electrical signals attendant to the depolarization and repolarization of a heart;
means for detecting an arrhythmia from the electrical signals;
means for discriminating between an atrial arrhythmia and a ventricular arrhythmia; and
means for drug delivery comprising a first drug pump means and a second drug pump means,
wherein the first drug pump means contains a first drug and the second drug pump means contains a second drug,
wherein the first drug pump means dispenses the first drug when the arrhythmia is atrial arrhythmia, and
wherein the second drug pump means dispenses the second drug when the arrhythmia is ventricular arrhythmia.

41. The system of claim 40, further comprising a pacing system means configured to provide pacing pulses to cardiac tissue in response to the electrical signals.

42. The system of claim 40, wherein the system is implantable in a human body.

43. The system of claim 40, further comprising controlling means configured to activate the first drug pump when the arrhythmia is atrial arrhythmia, and to activate the second drug pump when the arrhythmia is ventricular arrhythmia.
